(19) United States
(12) Patent Application Publication
(10) Pub. No.: US 2004/0265376 A1
(43) Pub. Date: Dec. 30, 2004

(54) Pharmaceutical compositions of 2'-deoxy-2'-(fluoromethylene) cytidine

(76) Inventor: Richard E. Jones, Palo Alto, CA (US)

Correspondence Address: Chiron Corporation, Intellectual Property - R440, P.O. Box 8097, Emeryville, CA, 94662-8097, US

(21) Appl. No.: 10/826,700

(22) Filed: Apr. 19, 2004

Related U.S. Application Data
(63) Continuation of application No. 09/867,770, filed on May 31, 2001, now abandoned.
(60) Provisional application No. 60/208,593, filed on Jun. 2, 2000.
(60) Provisional application No. 60/211,969, filed on Jun. 16, 2000.

(51) Int. Cl. A61K 31/52; A61K 9/20

(52) U.S. Cl. 424/464; 514/263.23

(57) ABSTRACT

Disclosed are orally deliverable pharmaceutical compositions of 2′-deoxy-2′-(fluoromethylene)cytidine (“FMdC”) and methods for providing enhanced bioavailability of FMdC in vivo.

### BACKGROUND OF THE INVENTION

1. Field of the Invention

This invention relates to orally deliverable pharmaceutical compositions of 2′-deoxy-2′-(fluoromethylene)cytidine (FMdC) and methods for providing enhanced bioavailability of FMdC in vivo.

Specifically, this invention relates to pharmaceutical compositions comprising FMdC which compositions are encapsulated in a material which does not dissolve at a pH less than 2 but readily dissolves at pH of about 4 to 5 or greater.

This invention also relates to methods for enhancing the oral bioavailability of FMdC in vivo by encapsulating FMdC in a material which does not dissolve at a pH of less than 2 but readily dissolves at a pH of about 4 to 5 or greater.

### REFERENCES

The following references are cited herein and are incorporated by reference in their entirety:

U.S. Pat. No. 5,378,693 to McCarthy et al., issued Jan. 3, 1995;

U.S. Pat. No. 5,508,393 to McCarthy et al., issued Apr. 16, 1996;

U.S. Pat. No. 5,589,587 to McCarthy et al., issued Dec. 31, 1996;

U.S. Pat. No. 5,595,979 to Snyder, issued Jan. 21, 1997;

U.S. Pat. No. 5,607,925 to Matthews et al., issued Mar. 4, 1997;

U.S. Pat. No. 5,616,702 to Edwards et al., issued Apr. 1, 1997;

U.S. Pat. No. 5,760,210 to McCarthy et al., issued Jun. 2, 1998;

U.S. Pat. No. 5,792,841 to Edwards et al., issued Aug. 11, 1998;

Handbook of Pharmaceutical Excipients, 1986, published by American Pharmaceutical Association, Washington, D.C., pages 251-252.

### STATE OF THE ART

2′-deoxy-2′-(fluoromethylene)cytidine (“FMdC”) is a nucleoside analog that is a ribonucleotide reductase inhibitor and a DNA chain terminator. Compounds that have these activities inhibit DNA synthesis. Accordingly, these types of compounds are useful for inhibiting cell growth and/or inhibiting viral replication. In view of these properties, FMdC has been suggested for use in the treatment of neoplastic diseases (cancer) and viral diseases. In the case of cancer, FMdC may be used either alone or in combination with radiation or chemotherapy. In the case of viral diseases, FMdC may be used either alone or in combination with other drugs.

When used for these purposes, the art teaches that FMdC can be administered to a patient either alone or in the form of a pharmaceutical composition in combination with pharmaceutically acceptable carriers or excipients. See, for example, Snyder, U.S. Pat. No. 5,595,979, and McCarthy, U.S. Pat. No. 5,378,693, both of which are incorporated by reference in their entirety. U.S. Pat. No. 5,378,693 notes that FMdC can be administered in a form or mode which makes the compound bioavailable in effective amounts, including orally. Oral administration is a preferred route of delivery.

Notwithstanding these teachings in the art, a problem has been encountered when the compound is administered in an oral form. Specifically, oral administration of this compound in a conventional tablet form resulted in less than desirable systemic uptake of the drug in the mammalian patient and a high patient-to-patient variability. That is to say that oral delivery did not provide for acceptable bioavailability of this drug.

After careful analysis, it was determined that FMdC lacks sufficient stability under acidic conditions to effectively traverse the acidic conditions of the stomach. The low bioavailability is attributed to degradation of the drug in the stomach. In addition, the observed marked patient-to-patient variability is attributed to differences in stomach emptying time.

Surprisingly, this lack of acidic stability is atypical of other members of this class of drugs. In fact, it has been found that FMdC is most stable at about pH 9. See, for example, FIG. 1 which illustrates FMdC's pH-stability profile and also demonstrates that at a pH of about 2 or less, this compound is very unstable. However, the pH of most mammalian stomachs can range to 2 or less and, at this pH, significant degradation of FMdC will occur.

Based on this discovery, this invention resides, in part, on administering FMdC in a form which can protect FMdC from acidic degradation arising from oral administration. However, merely protecting FMdC from acidic degradation is insufficient in obtaining maximal bioavailability for this drug. Specifically, bioabsorption initiates in the upper portions of the small intestine where the pH can be as low as about 4 to 5. Encapsulation of FMdC in materials which are resistant to acidic pH would result in undesirable loss of bioabsorption in this portion of the gastrointestinal tract.

Thus, for example, tablets or pills coated with sugar or shellac as coating agents, as disclosed in U.S. Pat. No. 5,378,693, would not be desirable. Specifically, a sugar coating is not acid stable and, accordingly, would dissolve in the stomach and expose FMdC to the acidic conditions of the stomach. This exposure to acidic conditions results in the degradation of the compound thereby reducing the bioavailability of FMdC. Shellac is insoluble in acidic conditions (e.g., pH 5) and is only soluble at alkaline pH and, accordingly, a coating of shellac would delay disintegration and drug release in the upper portions of the lower intestine thereby reducing bioabsorption of the drug. Further, studies using the USP disintegration test for shellac-coated tablets have indicated that there is a marked increase in the disintegration time over a six-month storage period for these tablets. It is likely that this effect is due to the polymerization of shellac which occurs over storage periods of this duration. See Handbook of Pharmaceutical Excipients, 1986, p. 251-252.

Thus, in spite of its established utility, the lack of high bioavailability when administered orally drastically reduces the effectiveness of the FMdC. Thus, it would be useful to provide a composition which would allow the safe delivery of FMdC to the small intestine where it can be absorbed into the blood stream.

### SUMMARY OF THE INVENTION

This invention is directed to orally deliverable pharmaceutical compositions of 2′-deoxy-2′-(fluoromethylene)cytidine (FMdC) and methods for providing enhanced bioavailability of FMdC in vivo. Specifically, this invention is directed to encapsulated 2′-deoxy-2′-(fluoromethylene)cytidine (FMdC) wherein the encapsulation material is selected to be dissolution resistant at a pH of 4 to 5 or less and to readily dissolve at a pH of greater than 4 to 5.

Accordingly, in one of its composition aspects, this invention is directed to an orally deliverable pharmaceutical composition comprising a pharmaceutically acceptable excipient or excipients and an effective amount of FMdC for treating a neoplastic disease or viral disease in a mammal wherein said composition is encapsulated in a material which is selected to be dissolution resistant at a pH of 4 to 5 or less and to readily dissolve at a pH of greater than 4 to 5.

In one embodiment, the pharmaceutically acceptable excipient or excipients comprise only the encapsulation material. In another embodiment, a separate pharmaceutically acceptable excipient and/or excipients are included in the encapsulation material.

Preferably, the encapsulation material is selected from the group consisting of cellulose acetate phthalate, hydroxypropyl methylcellulose phthalate, poly(vinyl acetate phthalate), hydroxypropyl methylcellulose acetate succinates, poly(meth)acrylates and cellulose acetate phthalate/diethylphthalate. More preferably, the encapsulation material is methacrylic acid:acrylic acid methyl ester copolymer.

Preferably, the composition comprises from about 50 to about 99.5 weight percent of the pharmaceutically acceptable excipient or excipients and from about 0.5 to about 50 weight percent of FMdC.

In one of its method aspects, this invention is directed to a method for enhancing the oral bioavailability of FMdC when orally delivered to a mammal which method comprises:

(a) encapsulating FMdC in a pharmaceutically acceptable material which is selected to be dissolution resistant at a pH of 4 to 5 or less and to readily dissolve at a pH of greater than 4 to 5; and

(b) orally delivering the product prepared in (a) above to said mammal.

### BRIEF DESCRIPTION OF THE DRAWING

FIG. 1 shows the pH-rate profile for the degradation of FMdC in Britton-Robinson buffer (pH 2 to pH 11) and 0.1N HCl (pH1) at 60° C.

### DETAILED DESCRIPTION OF THE INVENTION

This invention is directed to orally deliverable pharmaceutical compositions of 2′-deoxy-2′-(fluoromethylene)cytidine (FMdC) and methods for providing enhanced bioavailability of FMdC in vivo.

However, prior to discussing the invention in detail, the following terms are defined:

“FMdC” or “2′-deoxy-2′-(fluoromethylene)cytidine” refers to the compound represented by formula I:

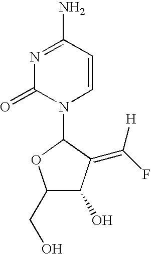

and pharmaceutically acceptable salts thereof.

This compound has alternately been named fluoromethylenedeoxycytidine; (E)-2′-deoxy-2′-(fluoromethylene)cytidine and (E)-2′-deoxy-2′-fluoromethylidenecytidine. Whether referred to by any of these names, each of these terms refers to FMdC.

The term “pharmaceutically acceptable salt” refers to pharmaceutically acceptable salts of FMdC, which salts are derived from a variety of organic and inorganic counterions well known in the art and include, by way of example only, salts of organic or inorganic acids, such as hydrochloride, hydrobromide, tartrate, mesylate, acetate, maleate, oxalate and the like.

The term “small intestine” or “upper intestine” refers to the part of the intestine that lies between the stomach and the colon. It consists of duodenum, jejunum and ileum and secretes digestive enzymes. It is the primary site of absorption of digested nutrients.

The term “neoplastic disease” refers to cancer, such as, hemaotological cancers such as leukemias and lymphomas; solid tumors such as carcinomas of the brain, head and neck breast, stomach, pancreas, kidney, liver, colon, ovaries, uterus, testicles, etc.; osteosarcomas, fibrosarcomas and Kaposi's sarcoma, etc.; which are characterized by uncontrolled or abnormal cell and/or tissue growth.

The term “viral disease” refers to hepatitis, HIV, cytomegalovirus (“CMV”), herpes virus, and influenza, etc.

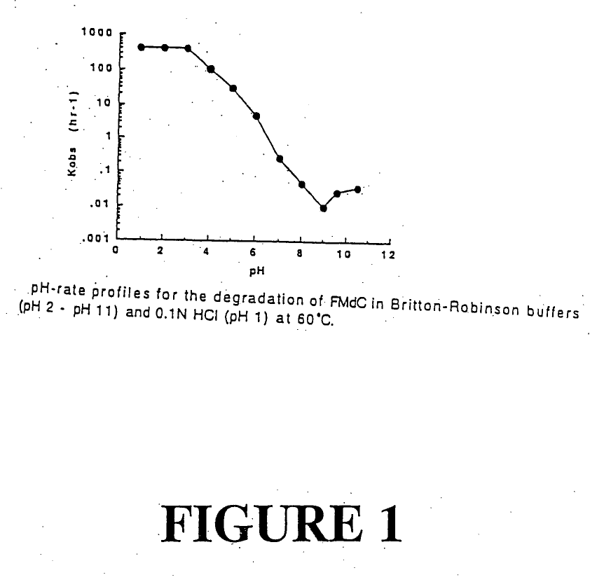

As discussed above, FMdC is known to be unstable and to degrade in acidic conditions such as those found in the stomach. FIG. 1 depicts the pH stability profile for FMdC, which shows that it is most stable at about pH 9. In order to achieve a high bioavailability of FMdC by oral administration, it is believed that FMdC which is encapsulated in a material which is selected to be dissolution resistant at a pH of 4 to 5 or less and to readily dissolve at a pH of about greater than 4 to 5 gives a higher bioavailability. When encapsulated in such material, the ingested dosage form of FMdC safely passes through the stomach and reaches the small intestine essentially intact where it then dissolves. Once the material coating the FMdC dosage form is dissolved, the FMdC is available for absorption into the blood stream through the small intestine. Surprisingly, notwithstanding the slightly acidic pH of the upper portions of the small intestine, the stability of FMdC at this pH is sufficient to allow enhanced bioavailability.

### PHARMACEUTICAL COMPOSITIONS

The compositions of this invention are achieved by using a pharmaceutical composition comprising FMdC encapsulated in a pharmaceutically acceptable material that dissolves in a pH of about 4 to 5 or more.

The compositions of this invention are first prepared in tablet, capsule or other suitable dosage form by methods well known in the art. In making the compositions of this invention, FMdC (active ingredient) is usually mixed with an excipient or excipients, diluted by an excipient(s), or enclosed within such a carrier which can be in the form of a capsule, tablet, granules, beads, pill, and the like. When the excipient(s) serves as a diluent, it is preferably a solid or semi-solid material, which acts as a vehicle, carrier or medium for the active ingredient. Thus, the compositions can be in the form of tablets, capsules, granules, beads, etc. containing, for example, up to 50% or more by weight of the active compound.

In preparing a formulation, it may be necessary to mill the active compound to provide the appropriate particle size prior to combining with the other ingredients. If a direct compression tablet formulation is to be used, milling of the active ingredient to a particle size of less than 200 mesh may be appropriate. If the active compound is substantially water soluble, the particle size is normally adjusted by milling to provide a substantially uniform distribution in the formulation, e.g. about 40 mesh, particularly if a granulation step is to be employed.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, and methyl cellulose. The formulations can additionally include: lubricating agents such as talc, magnesium stearate, and mineral oil; wetting agents; emulsifying and suspending agents; preserving agents such as methyl- and propylparaben; sweetening agents; flavoring agents and colorants.

The compositions are preferably formulated in a unit dosage form, each dosage containing from about 0.5 mg to about 500 mg, more usually about 1 mg to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, body surface area and response of the individual patient, the severity of the patient's symptoms, and the like.

Preferably, FMdC will be formulated into a composition containing a pharmaceutically inert carrier or carriers, including conventional solid carriers such as lactose, starch, dextrin, microcrystalline cellulose, mannitol, and the like, which composition is conveniently presented in the form of tablets, capsules, granules, beads, or the like.

For preparing solid compositions such as tablets, the principal active ingredient is mixed with a pharmaceutical excipient or excipients to form a solid preformulation composition containing a homogeneous mixture of the active ingredient. When referring to these preformulation compositions as homogeneous, it is meant that the active ingredient is dispersed evenly throughout the composition so that the composition may be readily subdivided into equally effective unit dosage forms such as tablets, capsules or packets of granules or beads. This solid preformulation is then subdivided into unit dosage forms of the type described above containing from, for example, 0.5 to about 500 mg of the active ingredient of the present invention.

The tablets, capsules, granules or beads of the present invention are then coated or otherwise compounded to provide a dosage form affording the advantage of stability at a pH of less than 2 and preferably less than 4 to 5.

The most preferred method for encapsulating FMdC is by enteric coating tablets, capsules, granules or beads containing FMdC which methods are well known in the art. Preferred materials for the enteric coating include, by way of example, cellulose acetate phthalate, hydroxypropyl methylcellulose phthalate, poly(vinyl acetate phthalate), hydroxypropyl methylcellulose acetate succinates, cellulose acetate phthalate/diethylphthalate, and, preferably, poly(meth)acrylates. The latter include copolymers of methacrylic acid and acrylic acid esters and/or methacrylic acid esters. When capsules are coated, a plasticizer should be used (such as hydroxypropyl methylcellulose acetate succinates/triethyl citrate or especially cellulose acetate phthalate/diethylphthalate) to minimize brittleness in the coating and to inhibit cracking of the coating. Tablets and granules can also be used.

Materials and compounds to enhance FMdC absorption may also be incorporated into the tablets, capsules, granules or beads.

Buffering agents may also be added to the tablets, capsules, granules, or beads in order to reduce the acidity of the immediate local environment of the small intestine and thus to maintain the stability of the FMdC so that it may be absorbed into the blood stream through the small intestine.

The following examples illustrate this invention.

### EXAMPLES

### Formulation Example 1

Hard gelatin capsules containing the following ingredients are prepared:

| | Quantity |
|---|---|
| Ingredient | (mg/capsule) |
| FMdC | 30.0 |
| Starch | 305.0 |
| Magnesium stearate | 5.0 |

The above ingredients are mixed and filled into hard gelatin capsules in 340 mg quantities. If necessary, the hard gelatin capsule is overcoated with a pharmaceutically acceptable material that does not dissolve until a pH of about 4 to 5 or more.

### Formulation Example 2

A tablet formula is prepared using the ingredients below:

| | Quantity |
|---|---|
| Ingredient | (mg/tablet) |
| FMdC | 1.0 |
| Cellulose, microcrystalline | 90.0 |
| Colloidal silicon dioxide | 6.0 |
| Stearic acid | 3.0 |

The components are blended and compressed to form tablets, each weighing 100 mg. The tablet is overcoated with a pharmaceutically acceptable material that does not dissolve until a pH of about 4 to 5 or more.

### Formulation Example 3

A tablet formula is prepared using the ingredients below:

| | Quantity |
|---|---|
| Ingredient | (mg/tablet) |
| FMdC | 5.0 |
| Cellulose, microcrystalline | 86.0 |
| Colloidal silicon dioxide | 6.0 |
| Stearic acid | 3.0 |

The components are blended and compressed to form tablets, each weighing 100 mg. The tablet is overcoated with a pharmaceutically acceptable material that does not dissolve until a pH of about 4 to 5 or more.

### Formulation Example 4

Tablets, each containing 30 mg of FMdC, are prepared as follows:

| | Quantity |
|---|---|
| Ingredient | (mg/tablet) |
| FMdC | 30.0 mg |
| Starch | 45.0 mg |
| Microcrystalline cellulose | 35.0 mg |
| Polyvinylpyrrolidone | 4.0 mg |
| (as 10% solution in water) | |
| Sodium carboxymethyl starch | 4.5 mg |
| Magnesium stearate | 0.5 mg |
| Talc | 1.0 mg |
| Total | 120 mg |

FMdC, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg. The tablet is then overcoated with a pharmaceutically acceptable material that does not dissolve until a pH of about 4 to 5 or more.

### Formulation Example 5

Capsules, each containing 40 mg of FMdC are made as follows:

| | Quantity |
|---|---|
| Ingredient | (mg/capsule) |
| FmdC | 40.0 mg |
| Starch | 109.0 mg |
| Magnesium stearate | 1.0 mg |
| Total | 150.0 mg |

The active ingredient, cellulose, starch, an magnesium stearate are blended, passed through a No. 20 mesh U.S. sieve, and filled into hard gelatin capsules in 150 mg quantities. If necessary, the hard gelatin capsule is overcoated with a pharmaceutically acceptable material that does not dissolve until a pH of about 4 to 5 or more.

1. An orally deliverable pharmaceutical composition comprising an effective amount of 2′-deoxy-2′-(fluoromethylene)cytidine for treating a neoplastic disease or viral disease in a mammal wherein said composition is encapsulated in a material which is selected to be dissolution resistant at a pH of about 4 to 5 or less and to readily dissolve at a pH of greater than about 4 to 5.

2. The orally deliverable pharmaceutical composition according to claim 1 further comprising a pharmaceutically acceptable excipient or excipients.

3. The orally deliverable pharmaceutical composition according to claim 2 wherein the pharmaceutically acceptable excipient or excipients comprise only the encapsulation material.

4. The orally deliverable pharmaceutical composition according to claim 2 wherein a separate pharmaceutically acceptable excipient or excipients is/are included in the encapsulation material.

5. The orally deliverable pharmaceutical composition according to claim 1 wherein the encapsulation material is selected from the group consisting of cellulose acetate phthalate, hydroxypropyl methylcellulose phthalate, poly(vinyl acetate phthalate), hydroxypropyl methylcellulose acetate succinates, poly(meth)acrylates and cellulose acetate phthalate/diethylphthalate.

6. The orally deliverable pharmaceutical composition according to claim 5 wherein the encapsulation material is selected from the group consisting of copolymers of methacrylic acid and acrylic acid esters and copolymers of methacrylic acid and methacrylic acid esters.

7. The orally deliverable pharmaceutical composition according to claim 2 wherein the composition comprises from about 50 to about 99.5 weight percent of the pharmaceutically acceptable excipient(s) and from about 0.5 to about 50 weight percent of 2′-deoxy-2′-(fluoromethylene)cytidine.

8. A method for enhancing the oral bioavailability of 2′-deoxy-2′-(fluoromethylene)cytidine when orally delivered to a mammal which method comprises:
(a) encapsulating 2′-deoxy-2′-(fluoromethylene)cytidine in a pharmaceutically acceptable material which is selected to be dissolution resistant at a pH of 4 to 5 or less and to readily dissolve at a pH of greater than 4 to 5; and
(b) orally delivering the product prepared in (a) above to said mammal.

9. A method of treating a neoplastic disease comprising administering to a mammal in need thereof a therapeutically effective amount of the pharmaceutical composition of claim 1.

10. The method of claim 9 further comprising administering radiation to the mammal.

11. The method of claim 9 further comprising administering a chemotherapeutic agent to the mammal.

12. A method of treating a viral disease comprising administering to a mammal in need thereof a therapeutically effective amount of the pharmaceutical composition of claim 1.

13. The method of claim 12 further comprising administering a second drug to the mammal.
